(19) United States
(12) Patent Application Publication
(10) Pub. No.: US 2007/0026444 A1
(43) Pub. Date: Feb. 1, 2007

(54) Thermal cycling in polymerase chain reactions by thermodynamic methods

(76) Inventor: Allan Heff, Newton, MA (US)

Correspondence Address: EDWARDS & ANGELL, LLP, P.O. BOX 55874, BOSTON, MA, 02205, US

(21) Appl. No.: 11/494,971

(22) Filed: Jul. 27, 2006

Related U.S. Application Data
(60) Provisional application No. 60/703,071, filed on Jul. 27, 2005.

(51) Int. Cl. C12Q 1/68; C12P 19/34

(52) U.S. Cl. 435/6; 435/91.2

(57) ABSTRACT

Systems and methods for rapid thermal cycling in a polymerase chain reaction (“PCR”). Thermodynamic work is performed, directly, or indirectly, or both, on or by analyte and reagents comprising a PCR mixture. The thermodynamic work, which is typically adiabatic, reversible, and isentropic, causes a rapid and uniform change in the temperature of the PCR mixture. Consequently, the denaturing, annealing, and extension steps in the PCR procedure may be performed rapidly, uniformly, and with greater efficiency and higher throughput.

### TECHNICAL FIELD

The present invention relates generally to methods and systems for performing a polymerase chain reaction and, more specifically, to methods and systems for thermal cycling in a polymerase chain reaction procedure.

### BACKGROUND INFORMATION

Polymerase chain reaction (“PCR”) is a commonly used amplification technique in molecular biology. It is frequently coupled with a detection technique to provide sensitive detection of trace amounts of nucleic acids. It has applications in clinical diagnostics, pharmaceutical development, life sciences research, and bio-defense. Important quality factors in PCR include speed, throughput, and reproducibility.

PCR consists of a repeated thermal cycling of a mixture of analyte and reagents. The analyte is typically DNA or RNA. The reagents include nucleic acid primers (e.g., oligonucleotide primers) and a high temperature polymerase. Each cycle has three stages: denaturing, annealing, and extension. The first stage, denaturing, occurs at high temperatures where the strands of the DNA separate. The second stage, annealing, occurs at a low temperature, where the probes and polymerase attach at a particular point to the denatured DNA strands. The third stage, extension, occurs at an intermediate temperature or at the low temperature, where the polymerase adds complementary nucleic acid base pairs to the DNA strand. Ideally, the number of gene copies doubles after each PCR cycle. Typically, thirty to forty thermal cycles are used. The temperatures used in the thermal cycles vary considerably. In some PCR systems, denaturing occurs between about 90 to 95 degrees C., annealing between about 55 to 60 degrees C., and extension between about 70 to 75 degrees C. In other systems, denaturing occurs between about 90 to 95 degrees C., and both annealing and extension occur at about 68 degrees C.

The speed of the PCR amplification is generally limited by several factors. These include the time for temperature ramps during thermal cycling, the time for the temperature throughout the PCR solution to come to equilibrium after a temperature ramp, the time in a single cycle for each of the three stages (denaturing, annealing, and extension), the number of cycles required for detection, and the amplification efficiency. Denaturing and annealing times are typically less than about one second. Extension time is proportional to the number of base pairs copied and can occur at a rate as fast as about one hundred base pairs per second.

Until recently, a typical turn-around time for PCR amplification was three hours. Recently, commercial devices have been produced with turn-around times as fast as thirty-five minutes.

Currently, most PCR thermal cycling is done by heat transfer using one of several methods: heat block, Peltier heat block, capillary tube, hot air, and flow-through. Each of these methods relies on the transfer of heat from a warm body to a cold body. Each of these methods is improved by the use of smaller sample size, larger sample surface to volume ratio, and thermally conductive sample container walls. Temperature change occurs initially at the walls and propagates into the bulk interior by diffusion, resulting in temperature heterogeneities in the sample, and degradation of the PCR amplification and specificity. Irradiating the sample with, for example, either microwave or infrared radiation can minimize this heterogeneous heating. If the sample is sufficiently small, all points of the sample will absorb heat homogeneously and temperature gradients are avoided. There is no equivalent mechanism for homogeneous cooling by heat transfer.

From the foregoing, it is apparent that there is a need for a method for rapid thermal cycling of analyte and reagents used in PCR. The rapid thermal cycling should uniformly heat and cool the analyte and reagents in accordance with the denaturing, annealing, and extension steps of the PCR. Because fast thermal cycling permits more amplifications per unit time, it provides a means for increased PCR throughput.

### SUMMARY OF THE INVENTION

The present invention provides methods and systems for rapid thermal cycling adapted for use with the PCR procedure. Methods according to the invention provide more rapid and homogeneous heating and cooling of analyte and reagents used in PCR in comparison to systems presently in use, with amplification potentially occurring in less than one minute.

The systems presently in use typically operate slowly, in part because their temperature cycling relies on the transfer of heat between bodies having different temperatures. In contrast, methods according to the invention produce temperature changes by performing thermodynamic work on the analyte and reagents, or on their container, or both, and generally without heat transfer. Because there is no heat transfer, the work is termed “adiabatic.” Consequently, the speed of the thermal cycling becomes limited by the kinetics of the PCR procedure. (Note that work performed on the container may be termed “indirect” work because any resulting changes in the condition of the container can affect the condition of the enclosed analyte and reagents (e.g., work on the container that raises its temperature generally causes a rise in the temperature of the contents of the container). This contrasts with “direct” work, which is performed on the analyte and reagents.)

The invention features a method for thermal cycling for PCR procedure, where adiabatic work is performed directly on a mixture of analyte and reagents (the “PCR mixture”), or indirectly on the container of the PCR mixture, or both, to change their temperature. The adiabatic work can include adiabatic compression, adiabatic stretching, or adiabatic polarization (electrical and magnetic). The resulting temperature change is rapid—typically occurring in less than about one second—and allows for denaturing of the PCR mixture once an appropriate temperature is reached.

In certain embodiments, further adiabatic work is performed to again change the temperature of the PCR mixture to allow for annealing and, optionally, extension. This further adiabatic work can include adiabatic expansion, adiabatic relaxation, or adiabatic depolarization (electrical and magnetic). In other embodiments, additional adiabatic work is performed to permit extension to occur at a temperature that is different from the annealing temperature.

Other embodiments of the invention include various apparatus to contain the PCR mixture and to allow adiabatic work to be performed. This adiabatic work can be performed on the PCR mixture, or on the container, or both. Also, it can be performed by the PCR mixture or by the container, or both. The apparatus can include a chamber with a movable piston, which permits adiabatic compression and decompression of the PCR mixture. Another configuration includes an elastomeric receptacle for receiving the PCR mixture. The elastomeric receptacle can be stretched and relaxed. Other configurations include chambers, receptacles, or carriers for the PCR mixture that permit electrical or magnetic polarization and depolarization.

Other aspects and advantages of the present invention will become apparent from the following detailed description, taken in conjunction with the accompanying drawings, illustrating the principles of the invention by way of example only.

### BRIEF DESCRIPTION OF THE DRAWINGS

The foregoing and other objects, features, and advantages of the present invention, as well as the invention itself, will be more fully understood from the following description of various embodiments, when read together with the accompanying drawings, in which:

FIG. 1 is a flowchart depicting a method for thermal cycling for a PCR procedure in accordance with an embodiment of the invention;

FIG. 2 is a flowchart depicting a method for thermal cycling for a PCR procedure in accordance with another embodiment of the invention;

FIGS. 3A, 3B, and 3C are schematic sectional views depicting a PCR chamber in accordance with an embodiment of the invention;

FIG. 4 is a schematic sectional view depicting a PCR chamber in accordance with another embodiment of the invention;

FIGS. 5A and 5B are schematic sectional views depicting a PCR chamber in accordance with another embodiment of the invention;

FIGS. 6A and 6B are schematic sectional views depicting a PCR chamber in accordance with another embodiment of the invention;

FIG. 7A is a schematic sectional view depicting a method for thermal cycling for a PCR procedure in accordance with another embodiment of the invention;

FIG. 7B is a schematic sectional view depicting a PCR chamber in accordance with another embodiment of the invention; and

FIGS. 8A and 8B are schematic sectional views depicting a PCR chamber in accordance with another embodiment of the invention.

### DESCRIPTION

As shown in the drawings for the purposes of illustration, the invention may be embodied in methods and systems for thermal cycling using adiabatic work. Embodiments of the invention are useful for rapid and homogeneous thermal cycling.

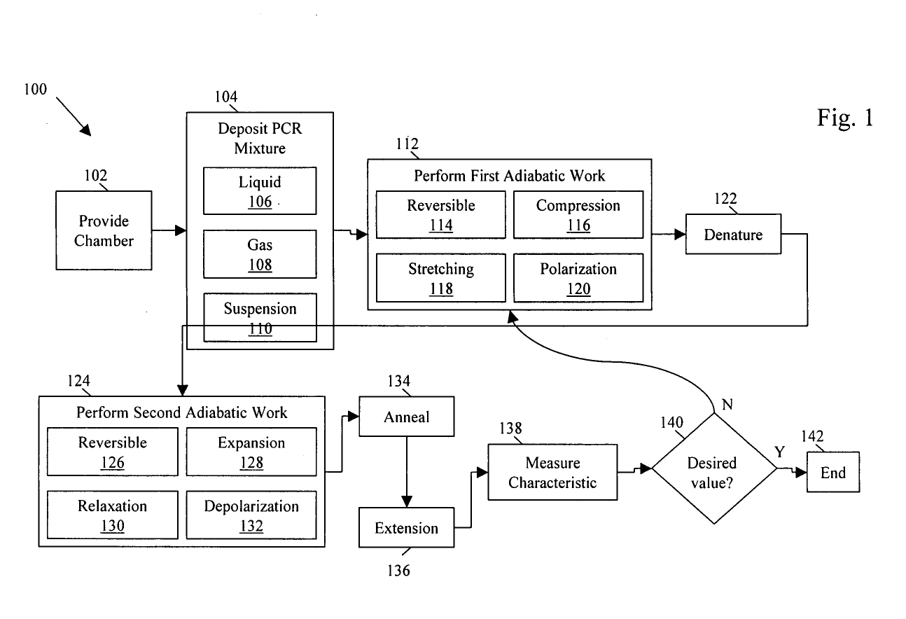

In brief overview, FIG. 1 is a flowchart depicting a method 100 for thermal cycling for a PCR procedure in accordance with an embodiment of the invention. The method includes a step of first providing a chamber (STEP 102) that will house the PCR mixture. Next, the PCR mixture is deposited into the chamber (STEP 104), which may be adiabatic. The PCR mixture can be in a liquid form 106, or gas form 108, or both (e.g., a liquid in equilibrium with its vapor). The PCR mixture can also be in the form of a suspension 110, for example, having small particles dispersed in a liquid.

Next, first adiabatic work is performed (STEP 112). This work, which may be reversible work 114, is typically performed on the chamber, or on the PCR mixture, or both. A result of performing the first adiabatic work is that the temperature of the chamber, or the PCR mixture, or both, increases. This typically occurs in less than about one second. In the case where the work is performed on the chamber, the temperature of the chamber rises and heat flows from the chamber to the PCR mixture by conduction or convection or a combination of both. This causes a rise in the temperature of the PCR mixture. In some embodiments, the work is performed on the PCR mixture, which also causes a rise in the temperature of the PCR mixture more directly than in the former case.

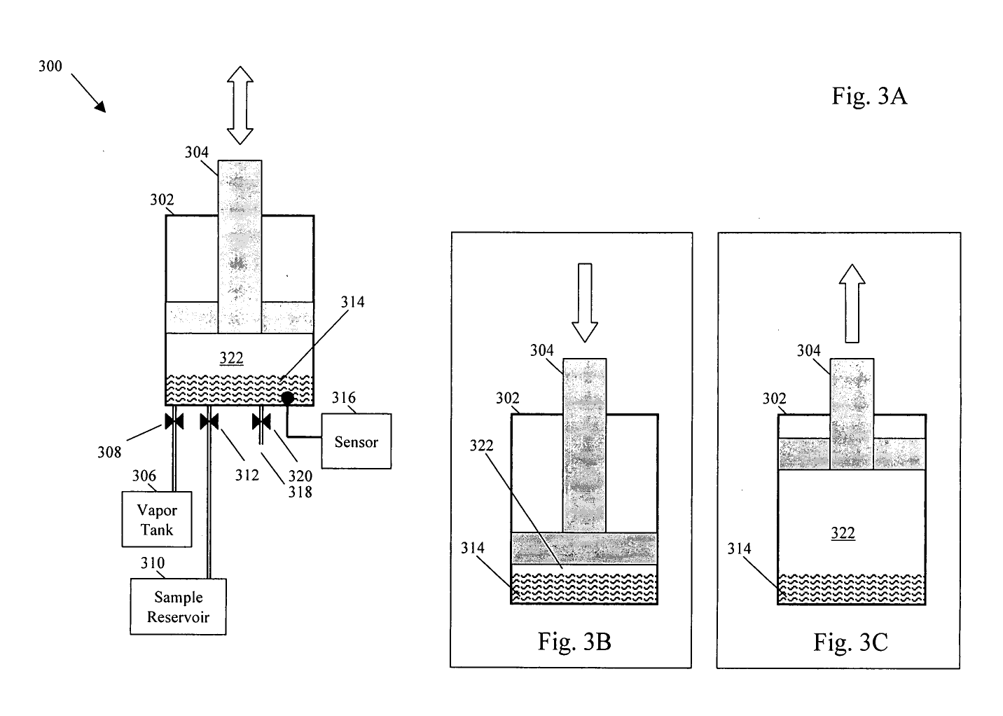

The first adiabatic work can be performed in a number of ways. In some embodiments, the first adiabatic work includes adiabatic compression of the PCR mixture (STEP 116). As discussed below in connection with FIGS. 3A, 3B, and 3C, the adiabatic compression includes reducing the volume of the chamber that contains the PCR mixture. As the volume is reduced, the pressure in and temperature of the PCR mixture increase according to thermodynamic principles. In the case where the PCR mixture includes a liquid in equilibrium with its vapor, this causes condensation of the vapor resulting in a rapid increase in temperature of the PCR mixture.

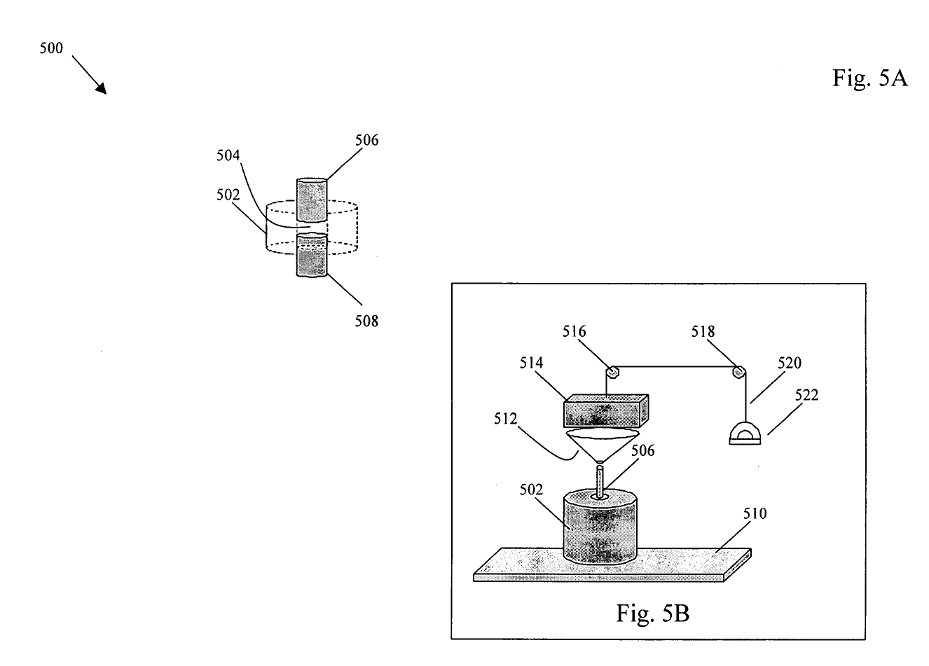

In other embodiments discussed below in connection with FIGS. 5A and 5B, the adiabatic compression is performed directly on the PCR mixture. In other words, the volume of the PCR mixture is reduced. This causes an increase in the pressure and temperature of the PCR mixture.

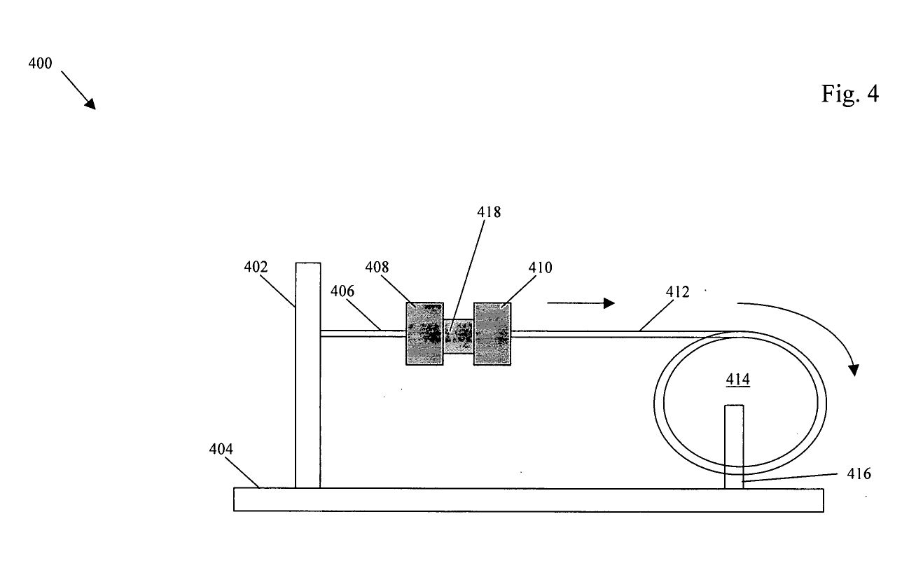

In another embodiment discussed below in connection with FIG. 4, the first adiabatic work includes adiabatic stretching (STEP 118) of the chamber. In this configuration, the chamber is typically constructed from an elastomer. Further, when stretched, tangled polymers in the elastomer become untangled, which results in the loss of entropy due to entanglement, and a rise in the temperature of the elastomer. Subsequent heat transfer from the elastomer to the PCR mixture warms the latter. In another embodiment, the surface area of the PCR mixture is increased by, for example, stretching a film of the PCR mixture. This increases the temperature of the film.

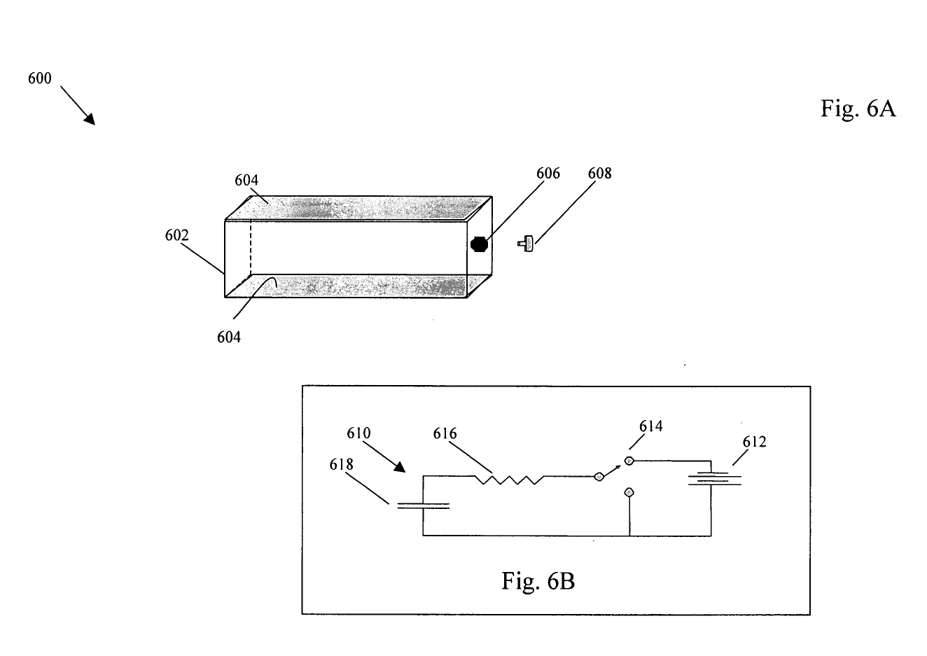

In other embodiments discussed below in connection with FIGS. 6A, 6B, 7A, 7B, 8A, and 8B, the first adiabatic work includes adiabatic polarization (STEP 120) of the chamber, or the PCR mixture, or both. The adiabatic polarization (STEP 120) can include adiabatic electrical polarization, or adiabatic magnetic polarization, or both. In any case, the adiabatic polarization causes a rise in the temperature of the PCR mixture.

Once the temperature of the PCR mixture rises to a desired level (e.g., between about 90 to 95 degrees C.), the method 100 includes the step of denaturing (STEP 122) the PCR mixture. During denaturing, high temperatures weaken molecular bonds causing the DNA double helix to separate into two single-stranded molecules. Denaturing typically occurs in about one second.

Following the denaturing step, second adiabatic work is performed (STEP 124). This work, which may be reversible work 126, is typically performed by the chamber, or by the PCR mixture, or both. A result of performing the second adiabatic work is that the temperature of the chamber, or the PCR mixture, or both, decreases. This typically occurs in less than about one second. In the case where the work is performed by the chamber, the temperature of the chamber falls and heat flows from the PCR mixture to the chamber by conduction or convection or a combination of both. This causes a drop in the temperature of the PCR mixture. In some embodiments, the work is performed by the PCR mixture, which also causes a drop in the temperature of the PCR mixture more directly than in the former case.

As with the first adiabatic work, the second adiabatic work can be performed in a number of ways. In some embodiments, the second adiabatic work includes adiabatic expansion of the PCR mixture (STEP 128). As discussed below in connection with FIGS. 3A, 3B, and 3C, the adiabatic expansion includes increasing the volume of the chamber that contains the PCR mixture. As the volume is increased, the pressure in and temperature of the PCR mixture decrease according to thermodynamic principles. In the case where the PCR mixture includes a liquid in equilibrium with its vapor, this causes evaporation of the liquid resulting in a rapid decrease in temperature of the PCR mixture.

In other embodiments discussed below in connection with FIGS. 5A and 5B, the adiabatic expansion is performed directly by the PCR mixture, thereby increasing the volume of the PCR mixture. This causes a decrease in the pressure and temperature of the PCR mixture.

In another embodiment discussed below in connection with FIG. 4, the second adiabatic work includes adiabatic relaxation (STEP 130) of the chamber. Similar to the configuration discussed above, the chamber is typically constructed from an elastomer and is in contact with the PCR mixture. When the elasomer chamber is relaxed, the chamber cools. Also, any drop in the temperature of the elastomer may cause heat transfer from the PCR mixture to the elastomer, thereby cooling the former. In another embodiment, the surface area of the PCR mixture is reduced by, for example, relaxing a film of the PCR mixture. This decreases the temperature of the film.

In other embodiments discussed below in connection with FIGS. 6A, 6B, 7A, 7B, 8A, and 8B, the second adiabatic work includes adiabatic depolarization (STEP 132) of the chamber, or the PCR mixture, or both. The adiabatic depolarization can include adiabatic electrical depolarization, or adiabatic magnetic depolarization, or both. In any case, the adiabatic depolarization causes a drop in the temperature of the PCR mixture.

Once the temperature of the PCR mixture drops to a desired level (e.g., between about 55 to 68 degrees C.), the method 100 includes the step of annealing (STEP 134) the PCR mixture. During annealing, oligonucleotide primers flank the target DNA and a polymerase binds to the single-stranded DNA. PCR typically uses a high temperature polymerase from the thermophilic bacterium Thermus aquaticus, called the Taq polymerase. This polymerase is a protein that combines strands of DNA together and is also capable of withstanding denaturing temperatures. Using current methods, annealing typically occurs in about one second.

In some embodiments, annealing is followed by an extension step (STEP 136) without changing the temperature of the PCR mixture. During extension, the polymerase extends the primers by adding complementary nucleic acid base pairs to the target DNA strands, thereby forming two double strands of the target DNA. The resulting copying of DNA can occur at a rate as fast as about one hundred base pairs per second. At the conclusion of the extension step a characteristic of the PCR mixture is measured (STEP 138), which is typically the number of DNA copies created. (Measurement of the characteristic at this point in the PCR procedure is sometimes referred to as “real time detection.”) If the desired number of DNA copies has been created (STEP 140), the method 100 ends (STEP 142) and the PCR mixture is generally removed from the chamber for further analysis. On the other hand, if the number of DNA copies is insufficient, the method 100 is repeated one or more times, beginning with the first adiabatic work (STEP 112), until the measured characteristic has the desired value (e.g., a sufficient number of DNA copies has been created). Typically, thirty to forty repetitions (i.e., cycles) may be needed to create a sufficient number of DNA copies.

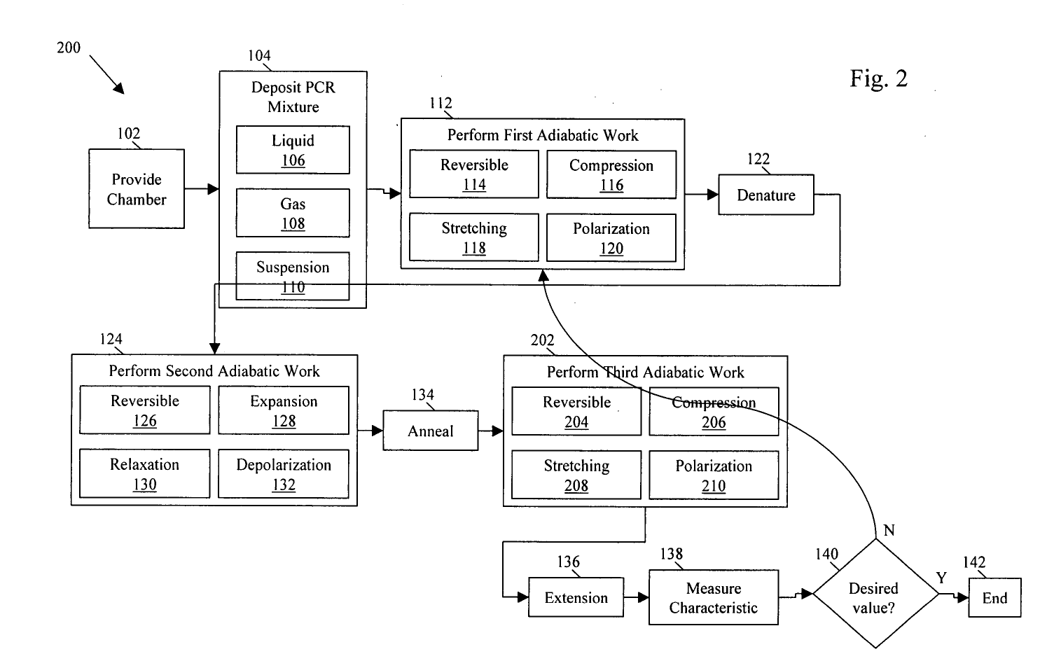

In other embodiments, a temperature change occurs between the annealing step (STEP 134) and the extension step (STEP 136). FIG. 2 is a flowchart depicting such a method 200, where the annealing step (STEP 134) is followed by the step of performing third adiabatic work (STEP 202). The third adiabatic work, which may be reversible work 204, is similar to the first adiabatic work described above. In particular, performing the third adiabatic work causes the temperature of the chamber, or the PCR mixture, or both, to increase, typically in less than about one second. The third adiabatic work may be performed on the chamber, or on the PCR mixture, or both. The third adiabatic work can include adiabatic compression of the PCR mixture (STEP 206), adiabatic stretching (STEP 208) of the chamber, and adiabatic polarization (STEP 210) of the PCR mixture. The adiabatic polarization (STEP 210) can include adiabatic electrical polarization, or adiabatic magnetic polarization, or both.

Once the temperature of the PCR mixture rises to a desired level (e.g., between about 70 to 75 degrees C.), the extension step (STEP 136) is performed, followed by the measurement of a characteristic of the PCR mixture (STEP 138). If the characteristic (e.g., the number of DNA copies) has the desired value (STEP 140), the method 200 ends (STEP 142) and the PCR mixture is generally removed from the chamber for further analysis. If the characteristic does not have the desired value, the method 200 is repeated one or more times, beginning with the first adiabatic work (STEP 112), which further raises the temperature of the PCR mixture, until the measured characteristic has the desired value. Typically, thirty to forty repetitions (i.e., cycles) may be needed to create a sufficient number of DNA copies.

In brief overview, FIGS. 3A, 3B, and 3C are schematic sectional views depicting a PCR apparatus 300 in accordance with an embodiment of the invention. The apparatus 300 is thermally insulated and includes walls 302 and a movable piston 304, which is typically low friction or frictionless. Generally, at least the walls 302 and the face of the movable piston 304 (which together define a chamber 322) are adiabatic. In some embodiments, the apparatus 300 has a cross section (i.e., face of the piston 304) area of about one square centimeter, and is at least about twenty-five centimeters long. The piston 304 fits snugly inside the apparatus 300 and moves freely over the length of the apparatus 300. The piston 304 may be moved manually or by using a mechanical actuator.

The walls 302 of the apparatus 300 and the piston 304 are constructed to withstand temperatures up to at least 100 degrees C. and internal pressures down to at least 0.2 atmospheres. The walls 302 of the apparatus 300 have low thermal conductivity and may be thick to limit the transfer of heat to the external environment. Many plastics are suitable for the walls 302 and piston 304, such as, for example, polycarbonate, nylon, and Teflon.

A vapor tank 306 is connected to the apparatus 300 via a vapor valve 308. The vapor tank 306 typically contains a two-phase mixture of liquid water and water vapor, held at a temperature between about 50 and 75 degrees C., and at a pressure of about 0.28 atmospheres. The level of water in the vapor tank 306 is sufficiently low such that only water vapor reaches vapor valve 308.

A sample reservoir 310 is connected to the apparatus 300 via a sample valve 312. The sample reservoir 310 contains at least 100 microliters of the PCR mixture 314. At least a portion of the PCR mixture 314 in the sample reservoir 310 is transferred to the apparatus 300.

One or more sensors 316 are typically located in the stationary end wall of the apparatus 300. In some embodiments, one or more of the sensors are located in recesses in the stationary end wall, and include a thermocouple, or a pressure transducer, or both. Leads from the sensors 316 generally extend through the wall of the apparatus 300 to one or more monitoring devices (e.g., meters). The thermocouple measures the temperature of the reaction mixture. Its temperature measurement range is generally about 20 to 100 degrees C. In some embodiments, the thermocouple is a standard k-type 0.005-in device. The pressure transducer measures the pressure of the chamber. Its pressure measurement range is generally about 0.1 to 1 atmosphere, absolute gauge.

A storage container (not shown) or a device for analysis of PCR products (not shown) may be connected to a port 318, which is connected to the apparatus 300 via a port valve 320. Typical devices for analysis include ethidium bromide-stained agarose gel electrophoresis, Southern blotting/probe hybridization, or fluorescence assay.

When the piston 304 is pushed into the apparatus 300, as shown in FIG. 3B, the volume of the chamber 322 is reduced. Conversely, when the piston 304 is pulled out of the apparatus 300, as shown in FIG. 3C, the volume of the chamber 322 is increased. In operation, the valves 308, 312, and 320 are initially closed and the piston 304 is pushed into the apparatus 300 such that the volume of the chamber 322 is minimum. Sample valve 312 to the sample reservoir 310 is opened and the piston 304 is pulled out of the apparatus 300 about one millimeter. This causes the apparatus 300 to fill with about 100 microliters of the PCR mixture 314. Sample valve 312 is then closed, and vapor valve 308 is opened. The piston 304 is pulled out of the apparatus 300 about twenty-five centimeters to increase the volume of the chamber 322 to about twenty-five milliliters. Consequently, the apparatus 300 fills with vapor, which, in some embodiments, is at a temperature of 68 degrees C. and at a pressure of approximately 0.28 atmospheres. Vapor valve 308 is then closed.

Next, the piston 304 is pushed in, reducing the volume of the chamber 322. As the volume of the chamber 322 decreases, the pressure rises, and since the PCR mixture 314 in the apparatus 300 is on the vaporization curve, droplets of liquid condense out of the vapor, which causes a rise in the temperature of the adiabatically isolated PCR mixture 314, and of any vapor in the chamber 322. The incremental temperature increase at any point on the vaporization curve may be calculated by use of the Clasius-Claperyon equation.

The piston 304 is pushed farther into the apparatus 300 until the volume of the chamber 322 is approximately 100 microliters. At this point, the resistance against the piston 304 rises considerably because all of the water vapor has condensed to liquid water. Optimally, the piston 304 moves its full length in less than a second. In some embodiments, after this compression, the temperature of the PCR mixture 314 is about 94 degrees C. and the pressure in the apparatus 300 is approximately 0.8 atmospheres. Denaturing of the PCR mixture 314 then occurs.

At the end of the denaturing step, the piston 304 is pulled out of the apparatus 300, increasing the volume of the chamber 322. As the volume of the chamber 322 increases, the pressure falls, and since the PCR mixture 314 in the apparatus 300 is on the vaporization curve, bubbles of vapor form in the PCR mixture 314, causing a fall in the temperature of the adiabatically isolated PCR mixture 314, and of any vapor in the chamber 322. The piston 304 is pulled farther out of the apparatus 300 until the apparatus 300 returns to its previous volume (twenty-five milliliters). Because this process is reversible, the system returns to its original state (i.e., to the initial temperature and pressure). Annealing, optionally followed by extension, of the PCR mixture 314 occurs at this temperature. Following the end of the annealing and optional extension steps, the cycle ends. (In other embodiments, the piston 304 is pushed back into the apparatus 300, which causes a rise in the temperature of the adiabatically isolated PCR mixture 314, and of any vapor in the chamber 322. Extension of the PCR mixture 314 can then occur at this higher temperature.)

Fast operation of the apparatus 300 requires brief reaction times at each step, rapid temperature ramps, and rapid thermal equilibrium at the end of each ramp. For a system already at the proper temperature, the reaction time for denaturing is generally less than one second. Similarly, for annealing, the reaction time is generally less than one second, and, for extension, the reaction time is approximately one to several seconds depending on target length. The ramp time is limited by the need for quasi-static motion, both of the piston 304 and of the PCR mixture 314, and of the vapor in equilibrium with the PCR mixture 314, between temperature states in the cycle, that is, both by frictionless travel of the piston 304, and by minimal dissipation in the PCR mixture 314. The process will be quasi-static for sufficiently slow piston motion, for example, if the motion is both frictionless and slow compared to the speed of sound in the PCR mixture 314, and of the vapor in equilibrium with the PCR mixture 314.

The apparatus 300 also allows for rapid thermal equilibrium. The changes in temperature due to evaporation and condensation propagate from the interfaces into the bulk of the PCR mixture 314. Nevertheless, in contrast to traditional heat exchanging, the distances involved are small, especially when vapor bubbles are interspersed throughout the PCR mixture 314. The time constants for thermal equilibrium in the PCR mixture 314 are generally less than one second. The number of bubbles may be increased by roughening the interior surface of the chamber 322 or by adding bubbling chips. Also, vapor bubbles move rapidly upward through the PCR mixture 314 because of the density differential. Hence, the temperature ramp and thermal equilibrium at the new temperature may be completed in as little as one-tenth of a second.

The thermal cycle described above is typically repeated thirty to forty times until amplification is complete. The apparatus 300 continues to cycle between the same temperature end-points as long as the process is isentropic, that is, as long as there is no heat transfer with the external environment and the process is quasi-static. Heat transfer is minimized by the use of thick, thermally insulating walls 302 and by the brevity of the thermal cycle, in particular, the brevity of the time spent during the cycle above the annealing and extension temperature(s).

After amplification, port valve 320 to an analysis device is opened and the piston 304 pushes the PCR mixture 314 out of the apparatus 300 for further analysis.

A variation to the embodiment described above includes a series of chambers 322 adapted for array pipetting. For example, an array of cylindrical wells (e.g., a micro-array or a micro-titer plate) is configured to receive samples of the PCR mixture 314. A corresponding array of pistons 304 is positioned such that each piston fits snugly inside each chamber. (The chambers are open at one end (i.e., the top) to receive the pistons 304. Further, these arrays are typically two-dimensional, i.e., having rows and columns of wells, and may be disposable to limit or eliminate cross-contamination.) The pistons 304 are generally configured to move in unison, and the cycling described above is able to occur in each cylindrical well in the array. This “simultaneous amplification” increases throughput of PCR amplifications. Transfer of the PCR mixture 314 in and out of the array of cylindrical wells can be accomplished by pipetting, which may be manual, robotic, or automatically controlled (e.g., under computer control). The arrays of cylindrical wells and pistons 304, and the samples of the PCR mixture 314 may be placed in an oven or environmental enclosure [a1]to facilitate further temperature or environmental control.

FIG. 4 depicts a different embodiment, wherein an apparatus 400 performs adiabatic work using adiabatic stretching and adiabatic relaxation. In brief overview, apparatus 400 includes a support wall 402 attached to a base 404. A support arm 406 is attached at one end to the support wall 402 and at the other end to a first clamp 408. A second clamp 410 is attached to one end of a flexible member 412, such as a cable or rope. The second end of the flexible member 412 is attached in a non-slip manner to a rotatable member 414, such as a drum, which is attached to the base 404 via a support column 416. In this configuration, the rotatable member 414 is able to rotate freely.

An elastomer 418 is disposed between and attached to the clamps 408, 410. The typical dimension of the elastomer 418 is three centimeters by one centimeter by one centimeter. Within the elastomer 418 is a well (i.e., void) for receiving the PCR mixture 314. The typical dimension of the well is ten millimeters by two millimeters by two millimeters. In operation, the PCR mixture 314 is deposited into the well. Next, the rotatable member 414 is rotated to place the flexible member 412 in tension. This stretches the elastomer 418 that is fixed between the clamps 408, 410. When the elastomer 418 is stretched, this causes a rise in the temperature of the elastomer, which causes the temperature of the PCR mixture 314 to rise. Once the temperature rises to an appropriate value, the denaturing begins.

Tension is maintained in the flexible member 412 until the end of the denaturing process, at which point the tension is removed by counter-rotation of the rotatable member 414. This allows the elastomer 418 to relax. Relaxation typically results in the elastomer 418 returning to its original temperature and length.

When the elastomer 418 is relaxed, this causes a drop in the temperature of the elastomer, which causes a drop in the temperature of the PCR mixture 314. When the temperature reaches a desired value, annealing, optionally followed by extension, occurs. In other embodiments, the flexible member 412 is again placed in tension by rotation of the rotatable member 414 to stretch the elastomer 418 and raise the temperature of the PCR mixture 314. Extension of the PCR mixture 314 can then occur at this higher temperature.

The thermal cycle resulting from the stretching and relaxation of the elastomer 418 is typically repeated thirty to forty times until amplification is complete. The repetitive, bidirectional rotation of the rotatable member 414 can be accomplished manually (e.g., by using weights) or electromechanically (e.g., by using a stepper motor). After amplification, the elastomer 418 is removed from the clamps 408, 410, and the PCR mixture 314 is removed from the well (e.g., by using a syringe) for further analysis.

FIGS. 5A and 5B depict an alternative embodiment, wherein an apparatus 500 performs adiabatic work using direct adiabatic compression and direct adiabatic decompression of the PCR mixture 314. In brief overview, apparatus 500 includes a thick walled cylinder 502 having an axial bore 504. A first piston 506 and a second piston 508 fit snugly in the bore 504. A void defined by the bore 504 and the faces of the pistons 506, 508 serves as a well to receive the PCR mixture 314.

The cylinder 502 and second piston 508 are disposed on a plate 510. Initially, piston 506 is removed and the PCR mixture 314 is deposited into the well by, for example, pipetting. The first piston 506 is then returned to the bore 504, and a cone 512 is attached to the first piston 506. A movable weight 514 is then placed on the cone 512, thereby compressing the PCR mixture 314. This causes an increase in the pressure and temperature of the PCR mixture 314. Once the temperature rises to an appropriate value, the denaturing begins.

The movable weight 514 is kept in position (i.e., on top of the cone 512) until the end of the denaturing process, at which point the movable weight 514 is lifted off of the cone 512. This results in the decompression of the PCR mixture 314, typically causing the PCR mixture 314 to return to its original temperature and pressure. When the temperature reaches a desired value, annealing, optionally followed by extension, occurs. In other embodiments, a different (e.g., smaller) movable weight 514 may be placed on the cone 512 to raise the temperature of the PCR mixture 314. Extension of the PCR mixture 314 can then occur at this higher temperature.

The thermal cycle resulting from the compression and decompression of the PCR mixture 314 is typically repeated thirty to forty times until amplification is complete. To sustain these repetitions, the cylinder 502, pistons 506, 508, plate 510, and cone 512 are generally constructed from a high tensile strength material (e.g., steel, beryllium, or copper). In any event, the repetitive movement of the movable weight 514 can be accomplished manually (e.g., by using a flexible member 520, pulleys 516, 518, and a handle 522) or electromechanically (e.g., by using a stepper motor). After amplification, the PCR mixture 314 is removed from the well (e.g., by using a pipette) for further analysis.

FIGS. 6A and 6B depict another embodiment wherein an apparatus 600 performs adiabatic work using adiabatic electrical polarization and adiabatic electrical depolarization of the PCR mixture 314. In brief overview, apparatus 600 includes a capacitive chamber 602 having electrodes 604 disposed on opposite exterior surfaces of the chamber 602. The electrodes 604 are connected to a power supply, typically via a switch. The capacitive chamber 602 includes an aperture 606 that may be closed using a cap 608.

FIG. 6B is a schematic depiction of an electrical model 610 of the apparatus 600. Capacitor 618 and resistor 616 represent the capacitive chamber 602. Power supply 612 is connected to the remainder of the circuit (i.e., the capacitive chamber 602) via a switch 614.

In operation, the PCR mixture 314 is deposited into the capacitive chamber 602 through the aperture 606 using, for example, a syringe. The aperture 606 is then sealed using the cap 608. The switch 614 is actuated to connect the electrodes 604 to the power supply 612. This charges the capacitor 618 (i.e., the capacitive chamber 602) and performs work on the PCR mixture 314. As a result, the PCR mixture 314 becomes electrically polarized and its temperature rises. Once the temperature rises to an appropriate value, the denaturing begins.

The electrodes 604 remain connected to the power supply 612 until the end of the denaturing process, at which point the switch is actuated to connect the electrodes 604 to ground. This discharges the capacitor 618 (i.e., the capacitive chamber 602). The discharging typically results in the PCR mixture 314 returning to its original temperature. Next, annealing, optionally followed by extension, occurs. In other embodiments, the switch 614 is again actuated to connect the electrodes 604 to the power supply 612 at a different (e.g., lower) voltage to raise the temperature of the PCR mixture 314. Extension of the PCR mixture 314 can then occur at this higher temperature.

The thermal cycle resulting from the charging and discharging of the capacitive chamber 602 is typically repeated thirty to forty times until amplification is complete. After amplification, the PCR mixture 314 is removed from the capacitive chamber 602 (e.g., by using a syringe) for further analysis.

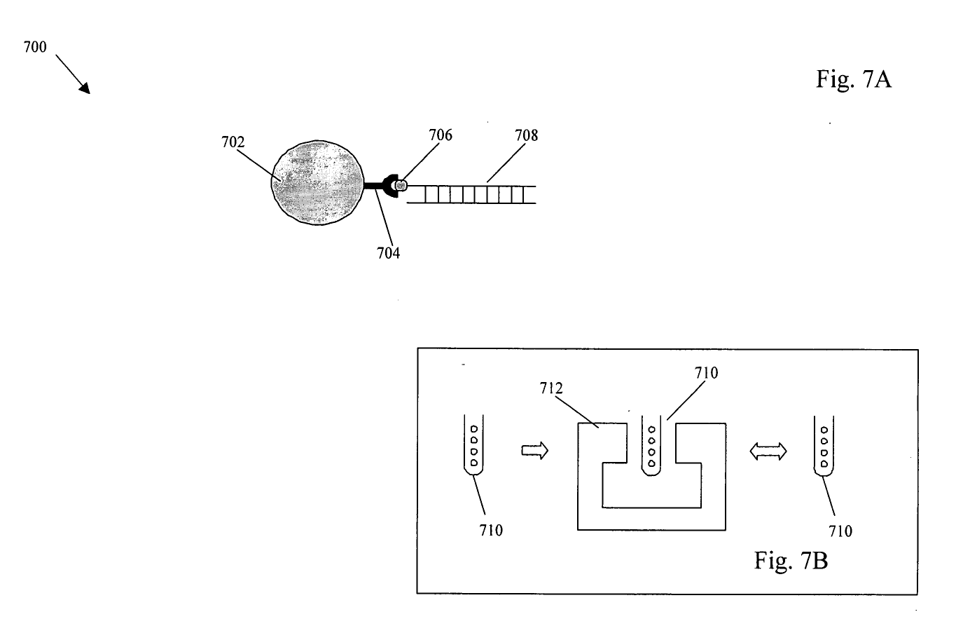

FIGS. 7A and 7B depict another embodiment 700 wherein target DNA 708, which is part of the PCR mixture 314, is captured on super-paramagnetic beads 702. The beads 702 are typically constructed from a super-paramagnetic highly polarizable material, such as gadolinium alloy Gd₅(SiₓGe₁₋ₓ)₄, where “x” is approximately 0.5. Then beads are then coated first with a polymer and second with a protein 704, such as streptavidin. A biotin 706 is attached to DNA fragments 708 in the PCR mixture 314, and the DNA fragments 708 then bind to the streptavidin-coated beads 702. Typically, contaminants are removed from the sample by placing the beads near a magnet; the beads are washed and only the DNA attached to the beads remain from the original sample. Then the beads 702, with the DNA fragments attached, are placed in a container 710 holding PCR reagents, resulting in a concentrated suspension. The container 710 is configured to be received in a rare earth permanent magnet 712. The magnetic field of the magnet 712 performs work on the beads 702 in the suspension, magnetically polarizing the beads 702. As a result, the temperature of the beads 702 and the surrounding PCR mixture 314 rises. Once the temperature rises to an appropriate value, the denaturing begins.

At the end of the denaturing process, the container 710 is removed from the magnet 712, causing the magnetic depolarization of the beads 702. This typically results in the beads 702 and PCR mixture 314 returning to their original temperature(s). Next, annealing, optionally followed by extension, occurs. In other embodiments, the container 710 is placed in another (e.g., weaker) magnet to raise the temperature of the beads 702 and PCR mixture 314. Extension of the PCR mixture 314 can then occur at this higher temperature.

The thermal cycle resulting from the magnetic polarization and depolarization of the beads 702 is typically repeated thirty to forty times until amplification is complete. After amplification, the beads 702 are used to transport the PCR mixture 314 to an analyzer.

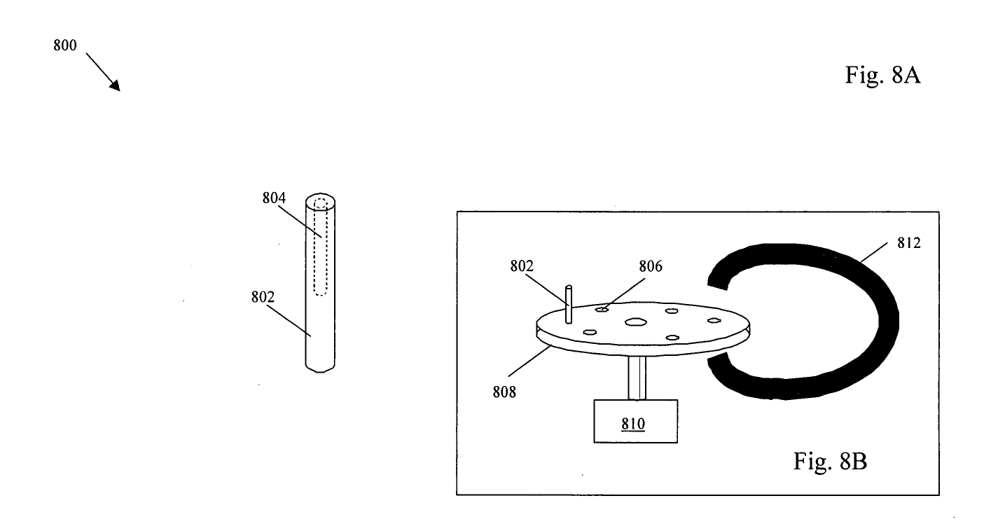

FIGS. 8A and 8B depict another embodiment 800 wherein the PCR mixture 314 is received in super-paramagnetic highly polarizable container 802. The typical outer dimension of the polarizable container 802 is five centimeters long by one centimeter in diameter. Within the polarizable container 802 is a well (i.e., void) 804 for receiving the PCR mixture 314. The typical dimension of the well is four centimeters long by 0.3 millimeter in diameter. Generally, the surface of the polarizable container 802 is coated with a thin layer of a non-reactive polymer.

After depositing the PCR mixture 314 in the well 804 (e.g., by using a syringe), the polarizable container 802 is received in a receptacle 806 on a turntable 808. Typically, the turntable 808 is constructed from a non-magnetic, electrically insulating material. Disposed around the turntable 808 are several C-shaped permanent magnets 812 that are constructed from, for example, neodymium-iron-boron. (For clarity, FIG. 8B shows only one magnet 812. In some embodiments, several magnets—as many as six, for example—are typically disposed around the turntable 808.)

In operation, a motor 810 slowly rotates the turntable 808. The polarizable container 802 becomes magnetically polarized when in passes through the magnetic field of the magnet 812. As a result, the temperature of the polarizable container 802 rises. Heat transfer from the polarizable container 802 to the PCR mixture 314 warms the latter. Once the temperature of the PCR mixture 314 rises to an appropriate value, the denaturing begins.

As the turntable 808 continues to rotate, the polarizable container 802 exits the magnetic field of the magnet 812. This magnetically depolarizes the polarizable container 802, which typically results in the polarizable container 802 returning to its original temperature. Heat transfer from the PCR mixture 314 to the polarizable container 802 cools the former. Next, annealing, optionally followed by extension, occurs. In other embodiments, the polarizable container 802 is again placed in a different (e.g., weaker) magnetic field of the magnet 812. (This occurs, for example, when several magnets 812 of varying strengths are disposed around the turntable 808 and the polarizable container 802 is moved into and out of magnetic fields due to the rotation of the turntable 808.) This raises the temperature of the polarizable container 802 and, by heat transfer, raises the temperature of the PCR mixture 314. Extension of the PCR mixture 314 can then occur at this higher temperature.

The thermal cycle resulting from the magnetic polarization and depolarization of the polarizable container 802 is typically repeated thirty to forty times until amplification is complete. After amplification, the PCR mixture 314 is removed from the well 804 (e.g., by using a syringe) for further analysis.

In various embodiments, a controller, such as, for example, a personal computer, is configured to monitor the temperature of the PCR mixture 314 and control the performance of the adiabatic work in response to the temperature. For example, the controller can change the volume of the chamber 322 as needed (e.g., by using an actuator) until the temperature PCR mixture 314 reaches the desired values for denaturing, annealing, and extension steps of the PCR procedure. Similarly, the controller can direct the application of electrical and magnetic fields to manage adiabatic polarization and depolarization to change the temperature PCR mixture 314. Also, the controller can manage the amount of time allowed for each step of the PCR procedure and, in the case of real time detection, measure the desired characteristic and determine whether additional PCR cycles are needed. Accordingly, the controller, in combination with sensors 316 and software, forms a closed loop system for automated control of a PCR procedure. The controller can also manage the movement of the PCR mixture 314 by controlling pipettes, syringes, and arrays using robotic devices. This could provide completely automated control of virtually all of the PCR procedure. Typically, the controller settings can be set by a user.

In a variation of the embodiments described above, at least a portion of one or more of the changes in temperature of the PCR mixture may be a result of ordinary heat flow into or out of the PCR mixture. Consequently, the entirety of the temperature changes need not be a result of only the adiabatic work.

The embodiments described above can be used in connection with ligase chain reaction (“LCR”) procedures. LCR is a DNA amplification technique based on the ligation of oligonucleotide probes. LCR differs from PCR because it amplifies the probe molecule rather than producing amplicon through polymerization of nucleotides. The probes are designed to match exactly two adjacent sequences of a specific target DNA. Like PCR, LCR requires a thermal cycler to drive the reaction and each cycle results in a doubling of the target nucleic acid molecule. LCR can have greater specificity than PCR.

LCR is repeated in three steps in the presence of excess probe material: (1) heat denaturation of double-stranded DNA; (2) annealing of probes to target DNA; and (3) joining of the probes by thermostable DNA ligase. After the reaction is repeated for about twenty to thirty cycles, the production of ligated probe is measured. (The production can also be measured after each cycle.) These steps are analogous to those performed in a PCR procedure as described above. Accordingly, methods and systems according to the invention, with the appropriate substitutions in, for example, reaction time, temperatures, and chemistry, generally can be used in LCR procedures.

The embodiments described above can be used in connection with gene sequencing.

From the foregoing, it will be appreciated that systems and methods according to the invention afford a simple and effective way to implement rapid and homogeneous thermal cycling in a PCR procedure.

One skilled in the art will realize the invention may be embodied in other specific forms without departing from the spirit or essential characteristics thereof. The foregoing embodiments are therefore to be considered in all respects illustrative rather than limiting of the invention described herein. Further, the phrase “at least one of” is intended to identify in the alternative all elements listed after that phrase, and does not require one of each element.

What is claimed is:

1. A method for thermal cycling for a polymerase chain reaction (PCR) procedure, the method comprising the steps of:
providing a chamber;
depositing a PCR mixture in the chamber; and
performing first adiabatic work, thereby changing a temperature of the PCR mixture from a first temperature to a second temperature.

2. The method of claim 1, wherein the chamber is adiabatic.

3. The method of claim 1, wherein the PCR mixture comprises a liquid.

4. The method of claim 1, wherein the PCR mixture comprises a gas.

5. The method of claim 1, wherein the PCR mixture comprises particles in suspension.

6. The method of claim 1, wherein the first adiabatic work comprises reversible work.

7. The method of claim 1, wherein the first adiabatic work comprises adiabatic compression of the PCR mixture.

8. The method of claim 7, wherein the adiabatic compression decreases a volume of the chamber and increases a pressure in the chamber.

9. The method of claim 1, wherein the first adiabatic work comprises adiabatic stretching of the chamber.

10. The method of claim 1, wherein the first adiabatic work comprises increasing a surface area of the PCR mixture.

11. The method of claim 1, wherein the first adiabatic work comprises adiabatic polarization.

12. The method of claim 11, wherein the adiabatic polarization comprises adiabatic electrical polarization.

13. The method of claim 11, wherein the adiabatic polarization comprises adiabatic magnetization.

14. The method of claim 1, wherein the first temperature is greater than the second temperature.

15. The method of claim 1, wherein the second temperature is greater than the first temperature.

16. The method of claim 1, wherein the step of changing the temperature of the PCR mixture from the first temperature to the second temperature occurs in less than about one second.

17. The method of claim 1, further comprising the step of denaturing the PCR mixture at the second temperature.

18-48. (canceled)

49. A method for thermal cycling for a polymerase chain reaction (PCR) procedure, the method comprising the steps of:
providing an adiabatic chamber;
depositing a PCR mixture in the chamber at a first temperature;
performing first adiabatic work, thereby raising the temperature of the PCR mixture from the first temperature to a second temperature;
performing second adiabatic work, thereby lowering the temperature of the PCR mixture from the second temperature to a third temperature; and
repeating the two performing steps in sequence until a desired characteristic is obtained for the PCR mixture.

50-72. (canceled)

73. A method for thermal cycling for a polymerase chain reaction (PCR) procedure, the method comprising the steps of:
providing an adiabatic chamber;
depositing a PCR mixture in the chamber at a first temperature;
performing first adiabatic work, thereby raising the temperature of the PCR mixture from the first temperature to a second temperature;
performing second adiabatic work, thereby lowering the temperature of the PCR mixture from the second temperature to a third temperature;
performing third adiabatic work, thereby raising the temperature of the PCR mixture from the third temperature to a fourth temperature; and
repeating the three performing steps in sequence until a desired characteristic is obtained for the PCR mixture.

74-96. (canceled)

97. A system for thermal cycling for a polymerase chain reaction (PCR) procedure, the system comprising:
an elastomeric chamber defining a cavity for receiving a PCR mixture, the elastomeric chamber having a first end and a second end; and
means for cyclically increasing and decreasing the distance between the first end and the second end, thereby changing a temperature of the elastomeric chamber.

98-103. (canceled)
